(19) United States
(12) Patent Application Publication
(10) Pub. No.: US 2008/0213865 A1
(43) Pub. Date: Sep. 4, 2008

(54) Compound microbial preparation

(75) Inventor: Wen-Hsiung Lai, Taichung City (TW)

Correspondence Address: TROXELL LAW OFFICE PLLC, SUITE 1404, 5205 LEESBURG PIKE, FALLS CHURCH, VA, 22041, US

(73) Assignee: Top Win Holding Company, LLC

(21) Appl. No.: 11/712,580

(22) Filed: Mar. 1, 2007

(51) Int. Cl. C12N 1/20; C12N 1/00; C12N 1/16

(52) U.S. Cl. 435/252.9; 435/243; 435/252.1; 435/252.4; 435/255.1

(57) ABSTRACT

A compound microbial preparation includes multiple single microbial series, hormones produced from each of those multiple single microbial series separately cultivated, and hormones produced from cross cultivation among those multiple single microbial series to be applied in modifying soil quality, activating soil, effectively degrading soil pollution, and helping growth of crops.

### BACKGROUND OF THE INVENTION

(a) Field of the Invention

The present invention is related to a compound microbial preparation, and more particularly, to one that is capable of modifying soil, activating soil, effectively degrading soil pollution, and helping crops grow.

(b) Description of the Prior Art

Facing destructed and contaminated ecological environment, and deteriorating physical/chemical properties of the soil, the farming industry is now caught in a predicament when most of the tillable farms are acidified, hardened, and plate massed and even suffer damages by fertilizers resulting in fertilizers failure, reduced soil activities, and sinking production value. Therefore, discarding the use of conventional fertilizer by redirecting efforts to develop microbial fertilizer has become a new topic of the farming industry in recent years. Microorganisms fix natural nitrogen fertilizer, convert ammonia and vulcanized hydrogen into fertilizer ingredients, dissolve phosphates otherwise insolvable in soil into phosphor fertilizer, and perform photosynthesis to produce glucose. Furthermore, microorganisms are capable of synthesizing organic fertilizer ingredients including anime and nucleic acid, decompose high polymer carbohydrates including fibers and starches into low polymer carbohydrates that facilitate growing of plants, and secret various types of organic acid, antibiotic substance, and growing hormone to promote plants to grow and improve their resistance to diseases. For example, bacterial including Thiobacillus, Nitrosomonas, and Nitrobacter found in phosphoric acid releasing bacteria of the good microbial series produce sulfuric acid and nitric acid to help dissolving phosphates found in the soil. Whereas these bacteria of good microbial series a part of the microbial community in the soil, providing a sound organic hotbed and a general soil microbial community is sufficient to loosen up the soil and correct acidified, hardened, and plate massed soil.

Operation of microorganisms to improve soil quality and growing of crops though appearing to be a modern trend, the technology not matured yet has been staying with the functional use of a single species of bacteria or a single microorganism resulting in flaws of low counts of live and effective bacteria, high level of foreign bacteria, and short effective term. Meanwhile, the farmers as the end users of microbial products do not understand correct way of application and application scope of bio-fertilizers. Consequently, for years, bio-fertilizer has never been given optimal use and recognition. Whereas the optimal use of a microorganism must compromise the adaptability of the regional ecology, i.e., the locality. Failure for a long time in screening for a matching strain that allows both of the strain and the region to adapt to each other has caused the use of bio-fertilizer to be at a standstill.

Therefore, there is the must to provide a pesticide preparation based on diversified compound microbial series from the good bacterial community to help plants growing while serving as the effective preparation to improve soil quality.

### SUMMARY OF THE INVENTION

The primary purpose of the present invention is to provide a compound microbial preparation that is applied in modifying soil quality, activating soil, effectively degrading soil pollution, and helping growth of crops.

To achieve the purpose, the present invention includes multiple single microbial series, hormones produced from each of those multiple single microbial series separately cultivated, and hormones produced from cross cultivation among those multiple single microbial series.

### BRIEF DESCRIPTION OF THE DRAWINGS

FIG. 1 is a schematic view showing a construction of microbial series of the present invention.

FIG. 2 is a schematic view showing a construction of crossing cultivation of microbial series of the present invention.

### DETAILED DESCRIPTION OF THE PREFERRED EMBODIMENTS

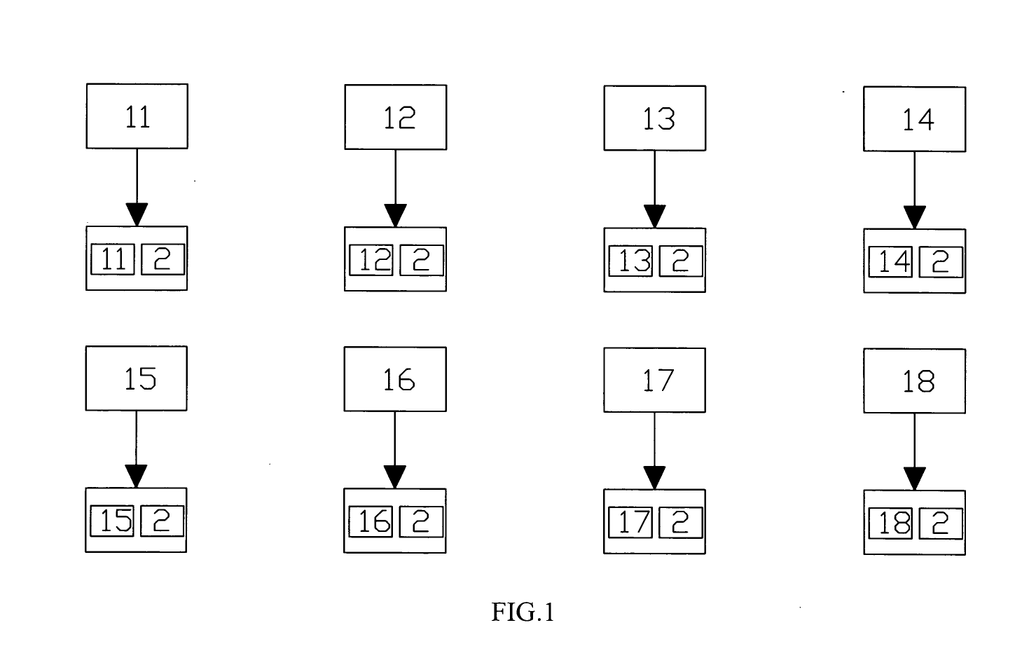

A compound microbial preparation of the present invention is formulated by having multiple single microbial series separately cultivated and followed with cross cultivation among those microbial series in a specific sequence and contains each of those microbial series, hormones produced from those single microbial series separately cultivated, and hormones produced by those single microbial series crossly cultivated for applications in modifying soil quality, activating soil, effectively degrading soil pollution, and helping growth of crops.

Wherein, those multiple single microbial series include nitrogen-fixing series 11, phosphoric acid releasing series 12, nitric acid series 13, photosynthetic bacterial series 14, lactobacillus series 15, yeast group series 16, actinomyces series 17, and growing factor producing series 18. As illustrated in FIG. 1, each microbial series during the process of individual cultivation produces through self-looping [Tricarboxylic acid (TCA) cycle] produces many active organics, which by nature are physiological organics other than allowing chelation and acid solution. Each single microbial series is separately cultivated in its designated cultivation medium, and the optimal pH in the growing and reproduction of different microbial series also varies. Therefore, proper control and regulation of pH of the cultivation medium must be provided in the course of bacterial cultivation and fermentation. The pH of the cultivation medium in the present invention is done with mono-potassium phosphate. While the growing and reproduction of the microbial series requires massive energy, the microbial series acquires the massive energy through aerobic respiration. However, the aerobic respiration generally has to rely upon only the oxygen dissolved in the cultivation medium, i.e., the dissolved oxygen, and the containment of the dissolved oxygen in the cultivation medium is not always provided in sufficient amount and will be soonest consumed by bacteria since oxygen is difficult to get dissolved in water. Therefore, constant air supply to the microbial series must be provided without interruption in the course of the cultivation and fermentation of the microbial series. Compositions of cultivation medium selected and the optimal growing environment conditions for each microbial series are detailed as follows:

1. A cultivation medium for the nitrogen fixing series 11 includes corn powder 6%, soybean powder 3%, corn steep liquor 1%, ammonium sulfate 0.4%, calcium chloride 0.5% and brown sugar 2%; a molasses (sugarcane) may be further added to function as an elicitor to facilitate fission; and the reproduction of the nitrogen fixing series 11 prefers an alkali (pH=8.5˜9.0) growing environment with a temperature range approximately of 20˜22° C. and produces many active organics 2. For example, the cultivation medium will secrete growing substances containing in ammoniac nitrogen, organic nitrogen, and amino acid.
2. A cultivation medium for the phosphoric acid releasing series 12 includes corn powder 4%, soybean cake powder 4%, sodium dihydrogen phosphate 0.6%, calcium chloride 0.4% and oligosaccharide 2%. The molasses (sugarcane) may be further added into the cultivation medium to function as an elicitor. Reproduction of phosphoric acid releasing series 12 prefers a growing environment of alkali (pH=8.5˜9.0) with a temperature range approximately of 20° C.˜22° C. and produces many active organics 2. For example, the series will secrete substances including organic acid, carbonic acid, and nitric acid to help the soil to undergo physical and chemical reactions to convert organic acid (e.g., nucleic acid, phospholipid) and non-solvable inorganic phosphor into soluble inorganic phosphor.
3. A cultivation medium for the nitric acid series 13 includes corn powder 6%, corn steep liquor 1%, fruit oligosaccharide 1%, and sodium dihydrogen phosphate (pH=7.2) 0.6%. β-galactose as an elicitor may be further added into the cultivation medium. Reproduction of the nitric acid series 13 prefers to reproduce in a mild acid (pH=6.0˜7.0) growing environment with a temperature range of 28˜32° C. and produces many active organics 2. For example, it will secrete active organics containing mycelium protein and amine.
4. A cultivation medium for the photosynthetic bacteria series 14 includes corn powder 6%, soybean cake powder 4%, corn steep liquor 0.8% and glucose 3.2%. Glucose may be further added into the cultivation medium to serve as an elicitor. Reproduction of the photosynthetic bacterial series 14 prefers to reproduce in a mild acid (pH=6.0˜7.0) growing environment with a temperature range approximately of 28˜32° C. and produces many active organics 2. For example, it will secrete general physiologically active substance including vitamins, Group B Vitamin, folic acid, biotin, coenzyme Q, virus resisting substances, and growth promoting factors; and nutrients needed for the proliferation of its microorganisms.
5. A cultivation medium for the lactobacillus series 15 includes corn powder 8%, soybean cake powder 4%, rice bran 1%, and tap water 6%. Lactose may be further added as an elicitor into the cultivation medium. Reproduction of the lactobacillus series 15 prefers an acid (pH=4.0˜6.0) with a temperature range approximately of 36˜38° C. and produces many active organics 2. For example, the lactobacillus series 15 will secrete lactobacillus-inhibiting factors, bacteria inhibiting molecules, and active organics with powerful decomposition strength.
6. A cultivation medium for the yeast group series 16 includes corn powder 12%, soybean cake powder 5%, bran 2% and tap water 5%. Lactose as an elicitor may be further added into the cultivation medium. Reproduction of the yeast group series 16 prefers an acid (pH=4.0-6.0) growing environment with a temperature range approximately of 36˜38° C. and produces many active organics 2. For example, it will secrete unicellular protein and active organic that promotes cell fission.
7. A cultivation medium for the actinomyces series 17 includes corn powder 8%, soybean cake powder 2%, bran 3.4%, sodium dihydrogen phosphate 0.4%, potassium dihydrogen phosphate 0.04%, and fruit oligosaccharide 1.2%. Glucose as an elicitor may be further added into the cultivation medium. Reproduction of the actinomyces series 17 prefers a neutral or mild alkali growing environment (pH=6.5˜8.0) with a temperature range approximately of 26˜28° C. and produces many active organics 2. For example, the actinomyces series 17 will secret active bacteria inhibiting substances including antibiotics, vitamins, and enzymes.
8. A cultivation medium for the growing factor producing series includes corn powder 10%, soybean powder 4%, amine chloride 0.1%, and glucose 1%. β-galactose as an elicitor may be further added into the cultivation medium. Reproduction of the growing factor producing series prefers a neutral or mild alkali (pH=6.5˜8.0) with a temperature range approximately of 26˜28° C. and produces many active organics 2. For example, the growing factor producing series will secrete fully loaded active growing substances including protein, antibiotics, and amino acid.

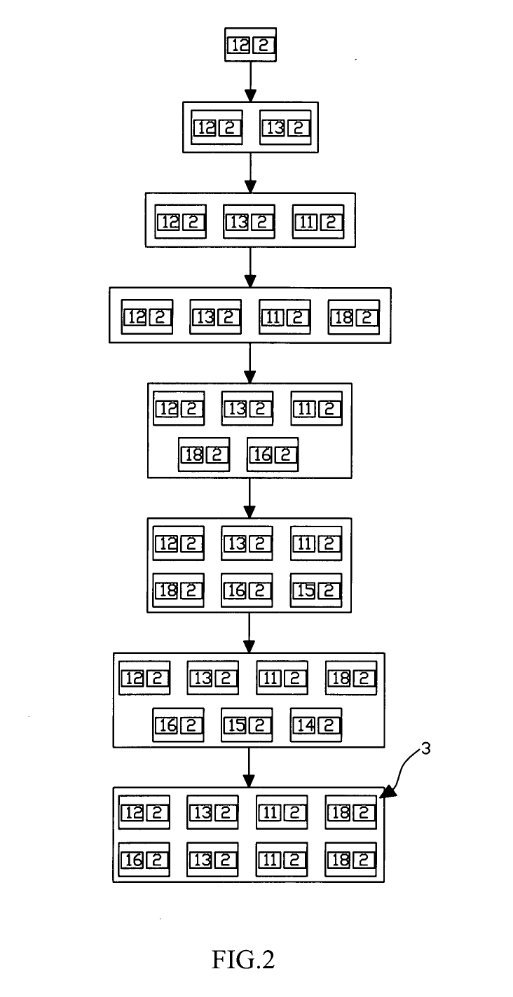

When the cultivation of each microbial series is saturated in its cultivation medium, a cross cultivation is followed as illustrated in FIG. 2. Wherein, the cross cultivation process is done in the sequence of the phosphoric acid releasing series 12, the nitric acid series 13, the nitrogen-fixing series 11, the growing factor producing series 18, the yeast group series 16, the lactobacillus series 15, the photosynthetic bacterial series 14, and actinomyces series 17 with addition ratios for each series respectively, 11.6% for the phosphoric acid releasing series 12, 8.2% for the nitric acid series 13, 12.3% for the nitrogen fixing series 11, 13.1% for the growing factor producing series 18, 16.7% for the yeast group series 16, 12.4% for the lactobacillus series 15, 8.4% for the photosynthetic bacterial series 14, 7.3% for the actinomyces series 17, and 10% for the fruit oligosaccharide to form a compound microbial preparation 3. In another composition, the compound microbial preparation 3 is comprised of 12.5% each of the phosphoric acid releasing series 12, the nitric acid series 13, the nitrogen-fixing series 11, the growing factor producing series 18, the yeast group series 16, the lactobacillus series 15, the photosynthetic bacterial series 14, and the actinomyces series 17, or in another composition yet, 25% each of the phosphoric acid releasing series 12, the lactobacillus series 15, the growing factor producing series 18, and the actinomyces series 17.

In the course of cross cultivation, each of those eight microbial series maintains intrigue symbiosis and shared prosperity among one another by playing an critical role with secretions of its own particular active organics. For example, the nitrogen fixing series converts the molecular nitrogen into ammoniac nitrogen and the resultant ammoniac nitrogen is partially to be consumed by the nitrogen fixing series, the remaining ammoniac nitrogen is synthesized into organic nitrogen to be consumed by other bacterial series; and the yeast group series may catalyze polysaccharide into simple sugar including glucose to be consumed by lactobacillus to convert into alcohol. Centering on the photosynthetic bacteria series and the yeast group series as leading cores, each microbial series supports activities of other microbial series with its synthetic proficiency while taking advantage of those substances produced by other microbial series to constitute a commonwealth circle. However, behind the big chain of food that relies upon symbiosis substances, a survival game of gigantic resistance and wipe out takes place among one another due to different properties. In the environment seeing violent stimulation, new endocrines are produced. What's more important is that any strain of bacteria survived is practically the top selected one with reliable activities.

Other than each single microbial series contained, the finish product of the compound microbial preparation also contains hormones categorized as follows:

A. Nutrition type hormone: including lytic and synthetic enzymes of protein, sugar, fat, and nucleic acid, and a wide range of nutrition derived enzymes;
B. Immune type hormone: multiple immune hormones (also known as antibiotics) including penicillin, erythromycin, Terramycin, and streptomycine secreted by each of those single bacteria series; and
C. Stimulating type hormone: including gibberellins (cell extending hormone), and cytokines.

It is to be noted that the compound microbial preparation of the present invention different from a single bacteria species or a single microbial product generally available in the market can be applied in soil modification. The present invention provides microbial life activities from multiple good microbial series that are mutually coordinated and contained for crops or plants to get the results of specific fertilizers; that is, multiple microorganisms are screened from the soil and tamed to become capable of improving nutrition of the crops, and then to provide nitrogen, phosphor, and potassium fertilizers essential to the growth of the plants in organic means by taking advantage of interaction among compound microbial preparations. Wherein, the nitrogen fixing series fixes nitrogen molecules in the nature to make it a nitrogen source for manufacturing fertilizers; the phosphoric acid releasing series unlocks and converts insolvable phosphates in the soil into phosphor, ferrous, and calcium fertilizers; the yeast group series makes it available in the making of vitamins and growing hormones, and decomposes organics to improve disease-resistant sufficiency of the plants; the photosynthetic bacteria series while being applied in manufacturing of glucose secrets carotenoid and eliminates toxic substances including hydrogen sulfide and ammonia; the actinomyces series secrets antibiotic substances at a constant amount on long-term bases to inhibit diseases; and the growing factors producing series also releases on long-term basic a given amount of growing hormones to promote roots, stalks and leaves of crops or plants to grow strong.

Depending on the locality, season, depth of soil, the present invention produces the proper strains of the microbial series. Those who are familiar with the art may apply on various series, e.g. coccus, bacillus, vibrio, or spirillum; different demands of oxygen, e.g., aerobic and/or anaerobic; different environmental requirements, e.g., acidophilus, alkalophilus, psycho-, meso-, or thermophilic to come up with a locality-specific compound microbial preparation and different microbial series may be used to produce compound microbial preparations in various applications, e.g., for fertilizer, pesticide, or promotion growth of flowers and fruits

The prevent invention provides a compound microbial preparation, and the application for a patent is duly filed accordingly. However, it is to be noted that the preferred embodiments disclosed in the specification and the accompanying drawings are not limiting the present invention; and that any construction, installation, or characteristics that is same or similar to that of the present invention should fall within the scope of the purposes and claims of the present invention.

I claim:

1. A compound microbial preparation includes having multiple single microbial series separately cultivated in different cultivation media; hormones produced from each of those single microbial series separately cultivated; and hormones produced by those single microbial crossly cultivated.

2. The compound microbial preparation as claimed in claim 1, wherein those multiple single microbial series includes nitrogen fixing series, phosphoric acid releasing series, nitric acid series, photosynthetic bacteria series, lactobacillus series, yeast group series, actinomyces series, and growing factors producing series.

3. The compound microbial preparation as claimed in claim 1, wherein each and all microbial series are given cross cultivation in the sequence of addition of the phosphoric releasing series, the nitric acid series, the nitrogen fixing series, the growing factors producing series, the yeast group series, the lactobacillus series, the photosynthetic bacteria series, and the actinomyces series.

4. The compound microbial preparation as claimed in claim 1, wherein the cross cultivation is performed according to the ratios of the phosphoric releasing series 11.6%, the nitric acid series 8.2%, the nitrogen fixing series 12.3%, the growing factors producing series 13.1%, the yeast group series 16.7%, the lactobacillus series 12.4%, the photosynthetic bacteria series 8.4%, the actinomyces series 7.3%, and fruit oligosaccharide 10%.

5. The compound microbial preparation as claimed in claim 1, wherein the cross cultivation is performed according to same addition ratio of 12.5% of each phosphoric releasing series, the nitric acid series, the nitrogen fixing series, the growing factors producing series, the yeast group series, the lactobacillus series, the photosynthetic bacteria series, and the actinomyces series.

6. The compound microbial preparation as claimed in claim 1, wherein the cross cultivation is performed according to a same addition ratio of 25% of the phosphoric acid releasing series, lactobacillus series, growing factors producing series, and the actinomyces series.

7. The compound microbial preparation as claimed in claim 1, wherein those hormones include nutrition, immune, and stimulating types of hormones.

8. The compound microbial preparation as claimed in claim 2, wherein a cultivation medium for the nitrogen fixing series includes corn powder 6%, soybean powder 3%, corn steep liquor 1%, ammonium sulfate 0.4%, calcium chloride 0.5% and brown sugar 2%.

9. The compound microbial preparation as claimed in claim 2, wherein a cultivation medium for the phosphoric acid releasing series includes corn powder 4%, soybean cake powder 4%, sodium dihydrogen phosphate 0.6%, calcium chloride 0.4% and oligosaccharide 2%.

10. The compound microbial preparation as claimed in claim 2, wherein a cultivation medium for the nitric acid series includes corn powder 6%, corn steep liquor 1%, fruit oligosaccharide 1%, and sodium dihydrogen phosphate (pH=7.2) 0.6%.

11. The compound microbial preparation as claimed in claim 2, wherein a cultivation medium for the photosynthetic bacteria series includes corn powder 6%, soybean cake powder 4%, corn steep liquor 0.8% and glucose 3.2%.

12. The compound microbial preparation as claimed in claim 2, wherein a cultivation medium for the lactobacillus series includes corn powder 8%, soybean cake powder 4%, rice bran 1%, and tap water 6%.

13. The compound microbial preparation as claimed in claim 2, wherein a cultivation medium for the yeast group series includes corn powder 12%, soybean cake powder 5%, bran 2% and tap water 5%

14. The compound microbial preparation as claimed in claim 2, wherein a cultivation medium for the actinomyces series includes corn powder 8%, soybean cake powder 2%, bran 3.4%, sodium dihydrogen phosphate 0.4%, potassium dihydrogen phosphate 0.04%, and fruit oligosaccharide 1.2%.

15. The compound microbial preparation as claimed in claim 2, wherein a cultivation medium for the growing factor producing series includes corn powder 10%, soybean powder 4%, amine chloride 0.1%, and glucose 1%.
